(19) United States
(12) Patent Application Publication
(10) Pub. No.: US 2023/0068990 A1
(43) Pub. Date: Mar. 2, 2023

(54) PLATELET STORAGE METHODS AND COMPOSITIONS

(71) Applicant: Platefuse, Inc., Cincinnati, OH (US)

(72) Inventors: Yi Zheng, Cincinnati, OH (US); Jose Cancelas, Cincinnati, OH (US)

(21) Appl. No.: 17/792,864

(22) PCT Filed: Jan. 13, 2021
(86) PCT No.: PCT/US2021/013315

§ 371 Date: Jul. 14, 2022

Related U.S. Application Data
(60) Provisional application No. 62/961,602, filed on Jan. 15, 2020.

(51) Int. Cl. A01N 1/02; C12N 5/078

(52) U.S. Cl. CPC A01N 1/0226; A01N 1/0284; C12N 5/0644

(57) ABSTRACT

Disclosed are compounds and compositions for slowing, preventing, or reversing platelet damage, particularly as may occur during blood banking or during refrigeration of platelets. Also disclosed herein are methods for storing platelets and methods for improving platelet survival upon transfusion with one or more compounds or compositions as described herein.

### CROSS REFERENCE TO RELATED APPLICATIONS

This patent application claims the benefit of priority to U.S. Provisional Application No. 62/961,602, filed Jan. 15, 2020. All of the foregoing applications are fully incorporated herein by reference in their entireties for all purposes.

### STATEMENT REGARDING FEDERALLY SPONSORED R&D

Financial support was provided in part by the following grants: NIH R01 HL147536, R43 HL123103, and UH54HL119810 (NCAI-CC).

### BACKGROUND

### Field

The present application relates to the fields of chemistry, biology, and medicine. Disclosed herein are chemical compositions and their methods of use. More particularly, disclosed herein are compounds, compositions for platelet storage, methods of platelet storage and methods of using such stored platelets.

### Description

Platelet transfusion is a common life-saving procedure to treat hemorrhage or to prevent bleeding in patients with low platelet counts or dysfunctional platelets, including cancer patients receiving chemotherapy. Recently, the application of large doses of platelets during resuscitation has been shown to significantly improve survival of trauma patients. Approximately 3 million doses of platelets are used in the USA alone each year. However, the current supply of platelets cannot effectively cope with the increased demand mainly because platelets, unlike other blood cells, can be stored only at room temperature but not in refrigeration.

Room temperature storage favors bacteria growth, and the risk of bacterial infection of platelet concentrate transfusion is estimated to be 50 times higher than that of refrigerated red blood cell products. Thus, the U.S. FDA limits platelet room temperature storage to up to 7 days, and actual shelf life is only 2.5-3 days with a growing list of FDA mandated tests for microbiological contamination after platelet collection from donors. Up to an average of 20% of platelet products are discarded due to expiration. Yet there are constant platelet shortages due to unpredictable increased usage in surgery, chemotherapies, and trauma situations. Short shelf life represents a major handicap to converting platelet products into effective commodities, and an effective method to safely store human platelets for an extended period of time is a hugely unmet medical need globally, which has been extensively studied but still without a solution.

Refrigerated platelets (1-6° C.), while hemostatically active, are rapidly cleared from circulation after transfusion, and the mechanism of this clearance system has been a longstanding mystery. The U.S. CFR allows for the use of cold platelets for up to 3 days of storage for trauma patients (21 CFR 640.24 and 640.25).

The development of a method to prevent platelet damage upon refrigeration for longer periods of time is a much needed, and long sought after advance in blood banking. Such development would revolutionize the current method of platelet storage.

### SUMMARY

Some embodiments provide a composition for platelet storage comprising platelets and a compound represented by the chemical structure of

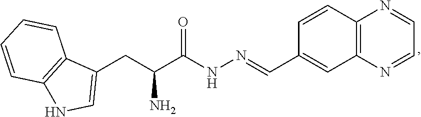 [SMILES: N[C@@H](Cc1c[nH]c2ccccc12)C(=O)N/N=C/c1ccc2nccnc2c1]

or a pharmaceutically acceptable salt thereof;

wherein the compound is present in an amount of about 1 μM to about 50 μM. In some embodiments, the compound is present in an amount of about 1 μM to about 20 μM. In some embodiments, the compound is present in an amount of about 1 μM to about 9 μM. In some embodiments, the compound is present in an amount of about 1 μM to about 5 μM.

Some embodiments provide a composition for platelet storage comprising platelets and a compound represented by the chemical structure of

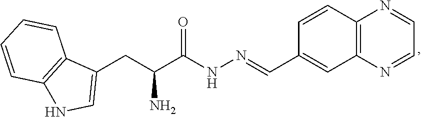 [SMILES: N[C@@H](Cc1c[nH]c2ccccc12)C(=O)N/N=C/c1ccc2nccnc2c1]

or a pharmaceutically acceptable salt thereof, wherein the (S) form of the compound is present in an enantiomeric excess of at least approximately 94%.

Some embodiments provide a composition for platelet storage comprising platelets and a compound represented by the chemical structure of

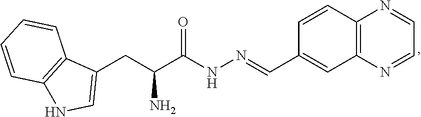 [SMILES: N[C@@H](Cc1c[nH]c2ccccc12)C(=O)N/N=C/c1ccc2nccnc2c1]

or a pharmaceutically acceptable salt thereof, wherein the amount of said compound in said composition is sufficient to achieve platelet survival and 24-hour recovery greater than about 65% of fresh peripheral blood platelets after 7 to 21 days of cold storage. In some embodiments, the compound is present in an enantiomeric excess of approximately 97%. In some embodiments, the composition further comprising a physiologically acceptable carrier. In some embodiments, the carrier is a buffer. In some embodiments, the carrier is selected from platelet additive solution (PAS), saline, phosphate buffered saline, Tris buffered saline, Hank's buffered saline, water, or a combination thereof. In some embodiments, the carrier comprises an electrolyte solution. In some embodiments, the composition comprises an additive selected from NaCl, KCl, CaCl2, MgCl₂, MgSO₄, Na₃ citrate, citric acid, NaHCO₃, Na phosphate, Na acetate, Na gluconate, glucose, maltose, mannitol, and combinations thereof. In some embodiments, the additive is present in an amount from about 0.5 mmol/L to about 150 mmol/L. In some embodiments, the additive includes one or more ingredients selected from D-ribose, D-glucose, Hanks solution, Hepes solution, bovine serum albumin, tic anticoagulant peptide and sterile water, or combinations thereof. In some embodiments, the additive is selected from pH adjusting and buffering agents, tonicity adjusting agents, stabilizers, wetting agents, and combinations thereof. In some embodiments, the composition has a pH from about 5 to about 8. In some embodiments, the composition is isotonic.

Some embodiments relate to a method for storing platelets comprising storing said platelets in a composition according to compound or compositions described herein. In some embodiments, the platelet survival is greater than about 65% after a storage period of 7-21 days of cold storage. In some embodiments, the storing is carried out at a temperature from about 0° C. to about 20° C. In some embodiments, the storing is carried out at a temperature from about 0° C. to about 10° C. In some embodiments, the storing is carried out at a temperature from about 1° C. to about 6° C. In some embodiments, the storing is carried out at a temperature of about 2° C.

Some embodiments described herein include a method for improving platelet survival upon transfusion. In some embodiments, the method includes contacting platelets with a composition of any one of compounds or compositions as described herein or the platelets stored according to the methods as described herein and infusing said contacted platelets into a subject. In some embodiments, the compound is present at a concentration from about 1 μM to about 20 μM. In some embodiments, the platelets are stored in the presence of the compound at a temperature of about 1° C. to about 25° C. In some embodiments, the platelets are stored in the presence of the compound for a period of about 1 to about 21 days. In some embodiments, the platelets are further contacted with a physiologically acceptable carrier.

### BRIEF DESCRIPTION OF THE DRAWINGS

FIG. 1A depicts the chemical structures of (S)G04 and (R)G04, and FIG. 1B is a graph showing the results of collagen-induced platelet aggregation studies of (S)G04 and (R)G04.

FIG. 2 is a graph showing the results of pharmacokinetic studies of (S)G04 in mice.

FIGS. 3A and 3B are graphs showing human platelet recovery after xenotransfusion of 7-day stored human platelets into humanized mice, pretreated with clodronate and sublethal irradiation (2.5 Gy).

FIGS. 4A and 4B are graphs showing human platelet recovery after xenotransfusion of 14-day stored human platelets into humanized mice, pretreated with clodronate and sublethal irradiation (2.5 Gy).

FIG. 5 is a graph showing the results of tail bleeding time of mice receiving platelet administration 24 hours after aspirin administration.

### DETAILED DESCRIPTION

Unless defined otherwise, all technical and scientific terms used herein have the same meaning as commonly understood by one of ordinary skill in the art to which the embodiments belong. Although any methods and materials similar or equivalent to those described herein may also be used in the practice or testing of the embodiments, the preferred methods and materials are now described. All publications mentioned herein are expressly incorporated by reference in their entireties.

### Definitions

It must be noted that as used herein and in the appended claims, the singular forms “a,” “an,” and “the” include plural referents unless the context clearly dictates otherwise. Thus, for example, reference to “a platelet” includes a plurality of such platelets and reference to “the carrier” includes reference to one or more carriers and equivalents thereof known to those skilled in the art, and so forth.

Where a range of values is provided, it is understood that the upper and lower limit, and each intervening value between the upper and lower limit of the range is encompassed within the embodiments.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

It is understood that the methods and formulations described herein include the use of pharmaceutically acceptable salts and/or conformers of compounds of disclosed embodiments, as well as metabolites and active metabolites of these compounds having the same type of activity. A conformer is a structure that is a conformational isomer. Conformational isomerism is the phenomenon of molecules with the same structural formula but different conformations (conformers) of atoms about a rotating bond. Likewise, it is understood that the compounds described herein, include the compound in any of the forms described herein (e.g., pharmaceutically acceptable salts, enantiomeric forms, tautomeric forms, and the like).

The term “pharmaceutical composition” refers to a mixture of one or more compounds disclosed herein with other chemical components, such as diluents or carriers. The pharmaceutical composition facilitates administration of the compound to an organism. Pharmaceutical compositions will generally be tailored to the specific intended route of administration.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C1-C7 alkylamine, cyclohexylamine, triethanolamine, ethylenediamine, and salts with amino acids such as arginine and lysine.

As used herein, a “carrier” refers to a compound that facilitates the delivery of a compound into cells or tissues. For example, without limitation, dimethyl sulfoxide (DMSO) is a commonly utilized carrier that facilitates the uptake of many organic compounds into cells or tissues of a subject.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

As used herein, the term “weight percent,” when referring to a component, is the weight of the component divided by the weight of the composition that includes the component, multiplied by 100%. For example the weight percent of component A when 5 grams of component A is added to 95 grams of component B is 5% (e.g., 5 g A/(5 g A+95 g B)×100%).

Composition

Refrigerated storage is believed to reduce platelet life-span due to decreased temperature that cause glycoprotein-Ib (GPIb) receptors to cluster on specific microdomains of the platelet membrane. Applicant has found that recognition of specific glycated/syalylated residues on clustered glycoproteins by macrophage β2 integrins and hepatocyte Ashwell-Morell receptors results in platelet phagocytosis by the host and removal from circulation. Thus, Applicant has identified prevention of glycoprotein clustering as a useful target for chemical intervention.

Platelet glycoproteins are intimately associated with intracellular cytoskeleton. Clustering of platelet glycoproteins depends on the formation of lipid raft in the platelet membrane, which in turn depends on the dynamics of the highly regulated processes of actomyosin assembly/disassembly. Rho family GTPases, including RhoA, Rac1 and Cdc42, are a class of GTP-binding enzymes that are central regulators of F-actin polymerization/depolymerization, and have been shown to control lipid raft formation and composition. Therefore, Applicant postulates that changes in Rho GTPase activities may influence platelet membrane lipid raft assembly and glycoprotein composition. Reversible targeting of Rho family GTPases by small molecule inhibitors may prevent cytoskeleton-dependent refrigeration storage lesions in platelets and result in increased platelet survival.

The mechanisms of how cold temperatures affect platelet survival are not completely understood, though significant information has been collected in the past decade. The effects of cold temperature on platelets are believed to be complex and involve shape change, cytoskeletal reorganization, activation, cell surface protein clustering and changes in the carbohydrate structures of surface glycoproteins. Refrigeration-induced changes including filopodia or lamellipodia are accompanied by an increase in the fraction of total cellular actin in a polymeric state (F-actin) and disappearance of a peripheral microtubule coil. Isolated prevention of microtubule polymerization using colchicine has not resulted in shape change prevention upon activation. Prevention of isolated actin dynamics using cytochalasin B results in reversion of discoid shape (18) but not in improved platelet survival in baboons, suggesting that irreversible blockade of actin polymerization does not prevent the refrigeration damage.

Presence of platelet cold receptors has been postulated as an explanation for both homeostatic and clinical effects when platelets are submitted to temperatures below 16° C. Cold temperature is believed to induce deglycosylation of glycoprotein Ib ectodomain exposing N-acetyl-D-glucosamine residues, which sequesters GM1 gangliosides in lipid rafts. Raft-associated glycoprotein Iba forms clusters upon binding of 14-3-3z adaptor proteins to its cytoplasmic tail, a process accompanied with mitochondrial damage and PS exposure (apoptosis-like)(24). The mechanisms of platelet clearance are believed to be associated with lipid-raft associated GPIb clustering and prevention of clustering prevents platelet clearance. Intimately associated with intracellular cytoskeleton, GPIb clustering depends on the formation of microdomains (so-called “lipid rafts”) in the platelet membrane which in turn depends on the dynamics of the highly regulated processes of acto-myosin assembly/disassembly at multiple levels.

Actin cytoskeletal rearrangements responsible for lipid rafts and GPIb clustering in lipid rafts depends on the coordinated activities of Cdc42, Rac1 and RhoA GTPases, which control specific downstream effectors in regulating polymerization and depoymerization of F-actin, actomyosin contraction, tubulin polymerization, and spectrin anchorage. The Rho family GTPases are a class of GTP-binding enzymes that act as signaling switches in spatial/temporal transduction and amplification of signals from platelet receptors to the intracellular signaling pathways that drive platelet function. Among the direct Rho GTPase effectors, WASPs, formins and PAKs that control F-actin polymerization/depolymerization have been shown to be crucial in the control of lipid raft formation and composition and tubular polymerization of platelets. Therefore, changes in Rho GTPase activities may influence platelet membrane microdomain assembly and glycoprotein composition. Earlier studies using dominant negative mutants of Cdc42 and Rac1 found no effect on prevention of cold-induced platelet damage (28), but the limitation of the tools used has prevented investigators from manipulating actin/actomyosin dynamics in a specific, reversible fashion.

Without intending to be limited by theory, it is believed that reversible inhibition of multiple Rho family of GTPases by chemical inhibitors can significantly improve platelet survival and transfusion function after refrigerated storage by interference with actomyosin dynamics and membrane microdomains to prevent GPIb clustering.

In one aspect, a composition for platelet storage is described herein. In some embodiments, the composition for platelet storage includes a compound represented by the chemical structure of:

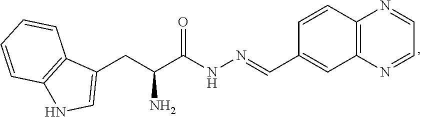 [SMILES: N[C@@H](Cc1c[nH]c2ccccc12)C(=O)N/N=C/c1ccc2nccnc2c1]

or a pharmaceutically acceptable salt thereof.

In some embodiments, the (S) form of the compound is present in an enantiomeric excess of at least approximately 60%, 65%, 70%, 75%, 80%, 85%, 90%, 91%, 92%, 93%, 94%, 95%, 96%, 97%, 98%, 99%, or ranges including and/or spanning the aforementioned values. In some embodiments, the (S) form of the compound is present in an enantiomeric excess of at least approximately 70%. In some embodiments, the (S) form of the compound is present in an enantiomeric excess of at least approximately 75%. In some embodiments, the (S) form of the compound is present in an enantiomeric excess of at least approximately 85%. In some embodiments, the (S) form of the compound is present in an enantiomeric excess of at least approximately 900%. In some embodiments, the (S) form of the compound is present in an enantiomeric excess of at least approximately 94%. In some embodiments, the (S) form of the compound is present in an enantiomeric excess of at least approximately 96%.

In some embodiments, the compound is present in an amount equal to or greater than: 0.1 μM, 1 μM, 2 μM, 3 μM, 4 μM, 5 μM, 6 μM, 7 μM, 8 μM, 9 μM, 10 μM, 12 μM, 14 μM, 15 μM, 16 μM, 18 μM, 20 μM, 22 μM, 24 μM, 25 μM, 26 μM, 28 μM, 30 μM, 32 μM, 34 μM, 35 μM, 36 μM, 38 μM, 40 μM, 42 μM, 44 μM, 45 μM, 46 μM, 48 μM, 50 μM, 52 μM, 54 μM, 55 μM, 56 μM, 58 μM, 60 μM, 62 μM, 64 μM, 65 μM, 66 μM, 68 μM, 70 μM, 72 μM, 74 μM, 75 μM, 76 μM, 78 μM, 80 μM, 82 μM, 84 μM, 85 μM, 86 μM, 88 μM, 90 μM, 92 μM, 94 μM, 95 μM, 96 μM, 98 μM, 100 μM, 105 μM, 110 μM, 11 μM, 125 μM, 130 μM, 140 μM, 15 μM, 160 μM, 170 μM, 180 μM, 190 μM, 200 μM, or ranges including and/or spanning the aforementioned values. Some embodiments, the compound is present in an amount of about 1 μM to about 100 μM, Some embodiments, the compound is present in an amount of about 1 μM to about 70 μM, Some embodiments, the compound is present in an amount of about 1 μM to about 20 μM, Some embodiments, the compound is present in an amount of about 1 μM to about 10 μM, In some embodiments, the compound is present in an amount of about 1 μM to about 9 μM. In some embodiments, the compound is present in an amount of about 1 μM to about 5 μM. Some embodiments, the compound is present in an amount of about 5 μM to about 100 μM, Some embodiments, the compound is present in an amount of about 5 μM to about 70 μM, Some embodiments, the compound is present in an amount of about 5 μM to about 20 μM, Some embodiments, the compound is present in an amount of about 5 μM to about 10 μM, In some embodiments, the compound is present in an amount of about 5 μM to about 9 μM. Some embodiments, the compound is present in an amount of about 10 μM to about 100 μM, Some embodiments, the compound is present in an amount of about 10 μM to about 70 μM, Some embodiments, the compound is present in an amount of about 10 μM to about 20 μM.

In some embodiments, the composition as described herein is sufficient to achieve platelet survival. In some embodiments, the composition as described herein is sufficient to achieve platelet function after storage. In some embodiments, the composition as described herein is sufficient to achieve platelet function after cold storage. In some embodiments, the composition as described herein is sufficient to improve platelet clearance. In some embodiments, the composition as described herein is sufficient to achieve platelet viability. In some embodiments, the composition as described herein is sufficient to achieve an improved platelet half-life. In some embodiments, the composition as described herein is sufficient to improve platelet viability. In some embodiments, the composition as described herein is sufficient to achieve in vitro platelet survival. In some embodiments, the composition as described herein is sufficient to achieve in vitro platelet survival in cold storage.

In some embodiments, platelet survival or function is greater than about 50%, 55%, 60%, 65%, 70%, 75%, 80%, 85%, 90%, 95%, or ranges including and/or spanning the aforementioned values, after storage. In some embodiments, platelet survival or function is greater than about 50% after storage. In some embodiments, platelet survival is greater than about 55% after storage. In some embodiments, platelet survival or function is greater than about 60% after storage. In some embodiments, platelet survival or function is greater than about 65% after storage. In some embodiments, platelet survival or function is greater than about 70% after storage. In some embodiments, platelet survival or function is greater than about 75% after storage. In some embodiments, platelet survival or function is greater than about 80% after storage. In some embodiments, platelet survival or function is greater than about 85% after storage.

In some embodiments, platelet survival or function may be maintained after a storage period. In some embodiments, the platelet survival or function may be maintained for a period of 1-7 days, 1-14 days, 4-7 days, 4-14 days, 7-14 days, 10-14 days, 7-21 days, 10-21 days, or ranges including and/or spanning the aforementioned values, of storage. In some such embodiments, the platelet survival or function may be maintained for a 24-hour recovery at greater than about 65%, greater than about 70%, greater than about 75%, greater than about 80%, greater than about 85%, greater than about 90%, greater than about 95% during cold storage, or ranges including and/or spanning the aforementioned values.

In some embodiments, the composition further comprises a physiologically acceptable carrier. In some embodiments, the carrier is a buffer. In some embodiments, the carrier is selected from platelet additive solution (PAS), saline, phosphate buffered saline, Tris buffered saline, Hank's buffered saline, water, or a combination thereof. In some embodiments, the carrier comprises an electrolyte solution. In some embodiments, the composition further comprises a pharmaceutically acceptable excipient. In some embodiments, the composition further comprises a stabilizer. Such materials should be non-toxic and should not interfere with the efficacy of the active ingredient. The pharmaceutically acceptable excipient, carrier, buffer, or stabilizer may take a wide variety of forms depending on the form of preparation desired for administration, e.g. intravenous or parenteral. In some embodiments, the PAS comprises at least one of sodium, potassium, magnesium, chloride, acetate, gluconate, glucose, HPO₄⁻², citrate, bicarbonate, calcium, or a combination thereof. In some embodiments, the PAS comprises a combination of sodium, potassium, magnesium, chloride, acetate, gluconate, glucose, HPO₄⁻², citrate, bicarbonate, and calcium. In some embodiments, the PAS comprises a combination of sodium with a range from about 130 to 200 mM, potassium with a range from about 0 to 5 mM, magnesium with a range from about 0 to 3 mM, chloride with a range from about 75 to 125 mM, acetate with a range from about 21 to 42 mM, gluconate with a range from about 0 to 23 mM, glucose with a range from about 0 to 30 mM, HPO₄⁻² with a range from about 0 to 30 mM, citrate with a range from about 0 to 15 mM, bicarbonate with a range from about 0 to 45 mM, and calcium with a range from about 0 to 1 mM. In some embodiments, the PAS has a pH with a range from about 5.4 to about 7.4.

In some embodiments, the amount of the carrier (in μM) is equal to or greater than about: 0.1, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 1000, 2000, 4000, 6000, 8000, 10,000, or ranges including and/or spanning the aforementioned values. In some embodiments, the concentration of the carrier in the composition (in μM) is equal to or greater than about: 0.1, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 1000, 2000, 4000, 6000, 8000, 10,000, or ranges including and/or spanning the aforementioned values.

In some embodiments, the amount of the carrier (in mg) is equal to or greater than about: 0.1, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or ranges including and/or spanning the aforementioned values. In some embodiments, the amount of the carrier present (in mg) is equal to or greater than about: 0.1, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or ranges including and/or spanning the aforementioned values.

In some embodiments, the weight percent of carrier in the composition is equal to or greater than about: 0, 1, 2, 5, 10, 20, 30, 40, 50, 60, 70, 80, or ranges including and/or spanning the aforementioned values.

In some embodiments, the composition further comprises an additive. In some embodiments, the additive may be selected from one or more pH adjusting and buffering agents, tonicity adjusting agents, stabilizers, wetting agents, and combinations thereof. In some embodiments, the additive may include one or more pH adjusting agents. In some embodiments, the additive may be selected from NaCl, KCl, CaCl₂, MgCl₂, MgSO₄, Na₃ citrate, citric acid, NaHCO₃, sodium phosphate, sodium acetate, sodium gluconate, glucose, maltose, mannitol, and combinations thereof. In some embodiments, the additive is present in an amount greater than 0.5 mmol/L, 1.00 mmol/L, 5 mmol/L, 10 mmol/L, 15 mmol/L, 20 mmol/L, 25 mmol/L, 30 mmol/L, 35 mmol/L, 40 mmol/L, 45 mmol/L, 50 mmol/L, 55 mmol/L, 60 mmol/L, 65 mmol/L, 70 mmol/L, 75 mmol/L, 80 mmol/L, 85 mmol/L, 90 mmol/L, 95 mmol/L, 100 mmol/L, 105 mmol/L, 110 mmol/L, 115 mmol/L, 120 mmol/L, 125 mmol/L, 130 mmol/L, 135 mmol/L, 140 mmol/L, 145 mmol/L, 150 mmol/L, 155 mmol/L, 160 mmol/L, 165 mmol/L, 170 mmol/L, 175 mmol/L, 180 mmol/L, 185 mmol/L, 190 mmol/L, 195 mmol/L, 200 mmol/L, or ranges including and/or spanning the aforementioned value. In some embodiments, the additive is present in an amount from about 0.5 mmol/L to about 150 mmol/L.

In some embodiments, the composition further comprises one or more ingredients selected from D-ribose, D-glucose, Hanks solution, Hepes solution, bovine serum albumin, tic anticoagulant peptide and sterile water, or combinations thereof.

In some embodiments, the composition has a pH from about 5, 5.5, 6.0, 6.5, 7.0, 7.5, 8, or ranges including and/or spanning the aforementioned values. In some embodiments, the composition is isotonic.

In some embodiments, the storage is carried out at a temperature greater than 0° C., 2° C., 4° C., 6° C., 8° C., 10° C., 12° C., 14° C., 16° C., 18° C., 20° C., 22° C., 24° C., 26° C., 28° C., 30° C., or ranges including and/or spanning the aforementioned values. In some embodiments, the storage is carried out at a temperature from about 0° C. to about 25° C. In some embodiments, the storage is carried out at a temperature from about 0° C. to about 20° C. In some embodiments, the cold storage is carried out at a temperature from about 0° C. to about 10° C. In some embodiments, the cold storage is carried out at a temperature from about 1° C. to about 6° C. In some embodiments, the cold storage is carried out at a temperature of about 2° C.

Some embodiments relate to a composition for platelet storage comprising platelets and a compound represented by the chemical structure of

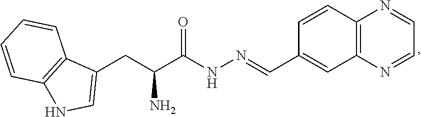 [SMILES: N[C@@H](Cc1c[nH]c2ccccc12)C(=O)N/N=C/c1ccc2nccnc2c1]

or a pharmaceutically acceptable salt thereof, wherein the amount of said compound in said composition is sufficient to achieve platelet survival and 24-hour recovery greater than about 65% of fresh peripheral blood platelets after 7-21 days of cold storage.

Some embodiments relate to a method for improving platelet survival upon transfusion comprising: contacting platelets with a compound of

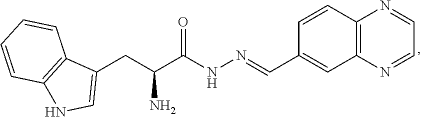 [SMILES: N[C@@H](Cc1c[nH]c2ccccc12)C(=O)N/N=C/c1ccc2nccnc2c1]

or a pharmaceutically acceptable salt thereof, and infusing said contacted platelets into a subject.

In some embodiments, the compound is present at a concentration from about 1 μM to about 20 μM. In some embodiments, the platelets are stored in the presence of the compound at a temperature of about 1° C. to about 25° C. In some embodiments, the platelets are stored in the presence of the compound for a period of 1 day, 2 days, 3 days, 4 days, 5 days, 6 days, 7 days, 8 days, 9 days, 10 days, 11, days, 12 days, 13 days, 14 days, 15 days, 16 days, 17 days, 18 days, 19 days, 20 days, 21 days, 22 days, 23 days, 24 days, 25 days, 26 days, 27 days, 28 days, or ranges including and/or spanning the aforementioned values. In some embodiments, the platelets are stored in the presence of the compound for a period of about 1 to about 14 days. In some embodiments, the platelets are further contacted with a physiologically acceptable carrier. In some embodiments, the platelets are stored in the presence of the compound for a period of about 4 to about 14 days. In some embodiments, the platelets are stored in the presence of the compound for a period of about 7 to about 14 days. In some embodiments, the platelets are stored in the presence of the compound for a period of about 10 to about 14 days. In some embodiments, the platelets are stored in the presence of the compound for a period of about 7 to about 21 days.

Some embodiments relate to a composition comprising a compound represented by the chemical structure

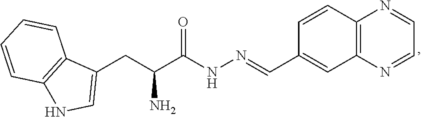 [SMILES: N[C@@H](Cc1c[nH]c2ccccc12)C(=O)N/N=C/c1ccc2nccnc2c1]

or a pharmaceutically acceptable salt thereof, and a platelet additive solution (PAS), wherein each 100 mL of PAS comprises: Sodium chloride 0.405 g, Potassium chloride 0.037 g, Magnesium chloride 6H₂O 0.030 g, acetate 3H₂O 0.442 g, Sodium citrate 2H₂O 0.318 g, Sodium dihydrogen phosphate 1H₂O 0.093 g, Disodium phosphate 12H₂O 0.769 g, and water for injection s.p. 100 mL.

Unless defined otherwise, all technical and scientific terms used herein have the same meaning as commonly understood by one of ordinary skill in the art to which the embodiments belong. Although any methods and materials similar or equivalent to those described herein may also be used in the practice or testing of the embodiments, the preferred methods and materials are now described. All publications mentioned herein are expressly incorporated by reference in their entireties.

Compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### EXAMPLES

### Synthesis of(S)G04

(S)G04 was synthesized according to the following scheme:

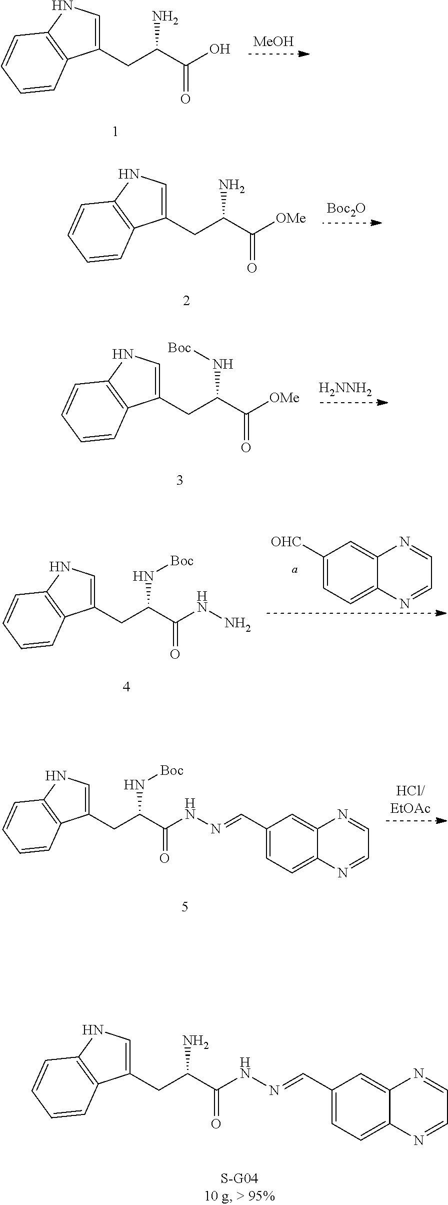

Synthesis yielded a pale yellow solid; NMR analysis confirmed the product as S-G04; LC/MS [M+H]⁺ was 359.30, with >95% purity. Chiral purity was 97%.

The above synthesis was carried out to achieve stereospecificity. Analogously, (R)G04 can be synthesized using D-tryptophan. Further, the method and procedures detailed in U.S. Pat. No. 10,028,503 to Zheng et al, issued Jul. 24, 2018, the contents of which are incorporated by reference for all purposes. One of skill in the art will recognize that analogous synthesis schemes may be used to synthesize similar compounds. One of skill in the art will recognize that compounds of the present embodiments may be synthesized using other synthesis schemes.

Inhibition of Collagen-Induced Platelet Aggregation

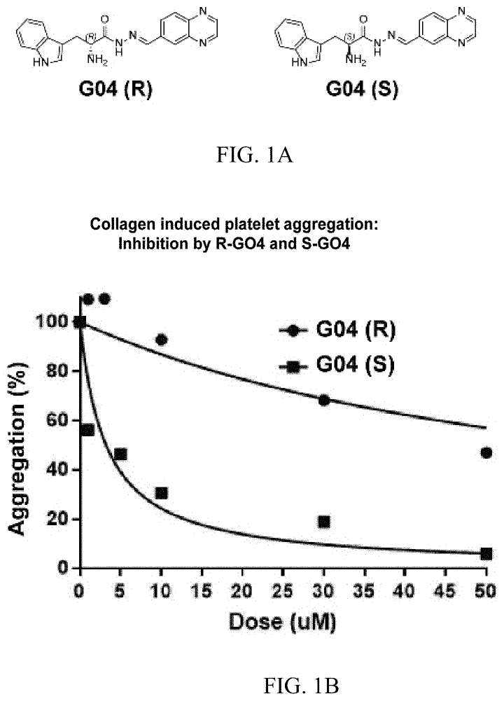

Platelet shape change in washed platelets was monitored using an aggregometer and the decrease in light transmittance following addition of an agonist (Huzoor et al., 1993). For the purpose of this analysis, collagen was used as the agonist. Briefly, a washed human platelet suspension was incubated at 37° C. in a Lumi-Aggregometer (Chrono-Log Corporation) with stirring at 900 rpm, followed by the addition of dimethylsulfoxide (DMSO) as a control vehicle, or DMSO containing (S)G04 or (R)G04 (FIG. 1A). (S)G04 and (R)G04 were tested at concentrations of 1, 5, 10, 30 and 50 μM.

After incubating the samples with the respective compound for 2 minutes, platelet aggregation was induced by adding collagen (1 μg/ml) and the relative aggregation value was read out at 6 min after the induction. A dose response for each compound was derived. These data were used to determine the IC₅₀ concentrations, at which 50% aggregation is reached.

The results of these experiments for (S)G04 and (R)G04 are shown in FIG. 1B. In these experiments, a collagen concentration of 4 μg/ml was used. The IC₅₀ values calculated for (R)G04 and (S)G04 are 40 μM and 4 μM, respectively.

Pharmacokinetic (PK) Studies of (S)G04

Standard (S)G04 was diluted from DMSO stock (50 mM) with PAS buffer into 10 and 50 μM, administered to mice via IV injection, and serum samples were collected at time points 1, 3, 10, 30 min, and stored at −80° C. before analysis. PK samples from mice serum (retro orbital bleed) were usually 20-100 μL.

A calibration curve in the range of 0-1 μM, i.e. 0, 0.01, 0.05, 0.1, 0.5, 1 μM, was prepared by spiking standard solution (10 M (S)G04 in PAS buffer) into blank mouse serum and then conducting serial dilution accordingly.

QC samples were prepared in the same way with final concentration of 0.05 and 0.5 μM.

Samples were prepared by carrying out the following steps. Add 20 μL of mouse serum (PK samples, calibrators and QCs), and 20 μL of internal standard in methanol, vortex for 30 secs. Add 100 μL of ice cold MeOH then vortex for 1 min. Let sit for 5 minutes before submitting to centrifuge for 10 minutes at 21,100×g (RCF) at 4° C. Take 100 μL of the supernatant and transfer to autosampler vial (max recovery, Waters). Put a cap on the vial, and load vials onto UPLC-MS autosampler for analysis. Because organic solvent evaporates relatively quickly at room temperature, samples in organic solvents should not be left at room temperature longer than 6 hr before injection. Set up the UPLC-MS/MS system: cleaning cone, in accordance with the manufacturer's Operation SOP/documents. The injection volume on the UPLC-MS/MS system is normally between 3-10 μl. Run sample on UPLC-MS/MS using the following parameters.

Solvent A: water 0.1% formic acid

Solvent B: acetonitrile 0.1% formic acid

The injection volume was 5 μL in positive mode

A gradient mobile phase was used with a binary solvent system, which changed from 95% solvent A to 0% solvent A over 5 min, hold for 3 min, then to 95% solvent A at 8.1 min, and this was held until 11 min. The total run time was 11 min, and the flow rate was 0.2 mL/min.

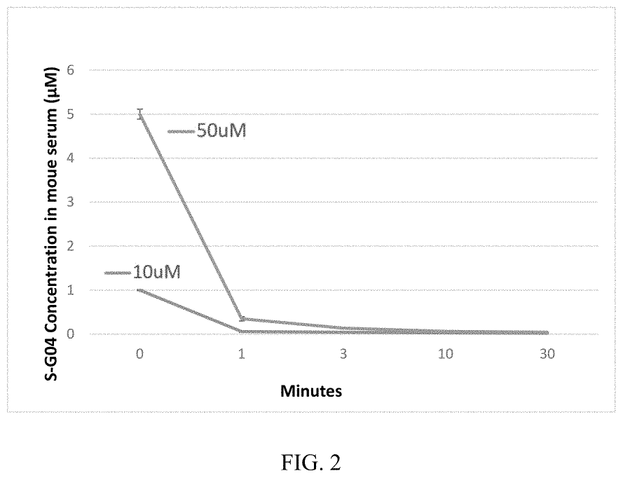

Column temperature was kept at 30° C. The capillary voltage is 1 KV for ES+ mode, cone voltage is 35 V, collision 25 V, desolvation temperature, 350° C.; desolvation gas flow, 800 L/h; source temperature, 120° C. The results of these experiments are depicted in FIG. 2 (upper line is 50 μM, lower line is 10 μM). These results demonstrate substantial reduction in (S)G04 concentration after 1 minute, and continued subsequent reduction. (S)G04 has a fast PK: >100× reduction of the injection concentration in 1 minute.

Human Platelet Recovery Studies

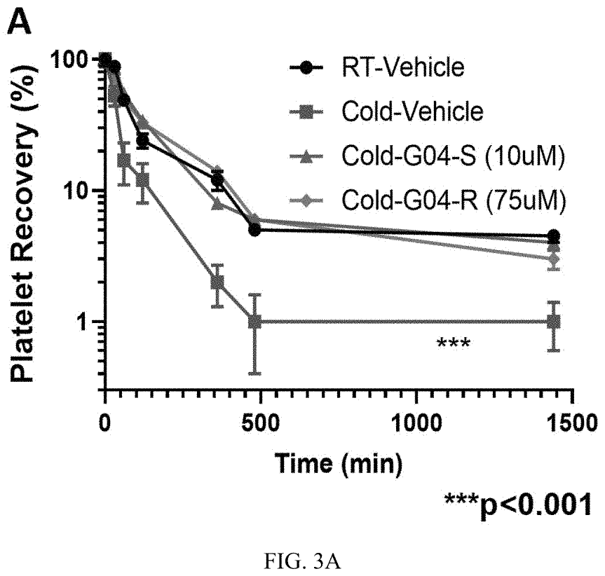

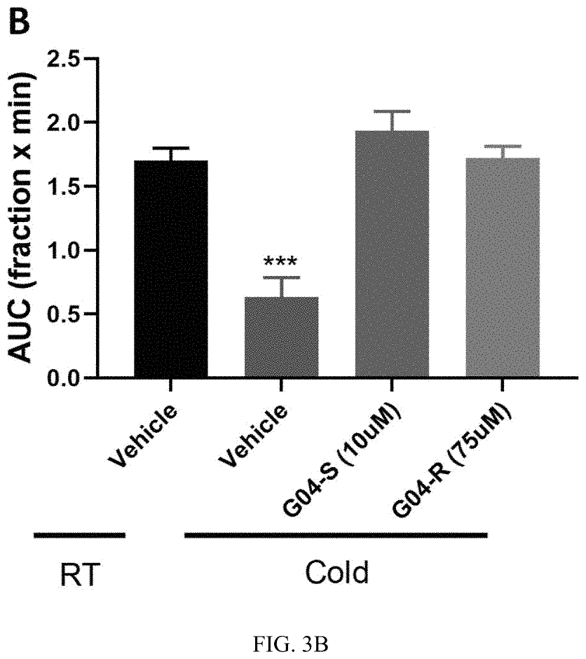

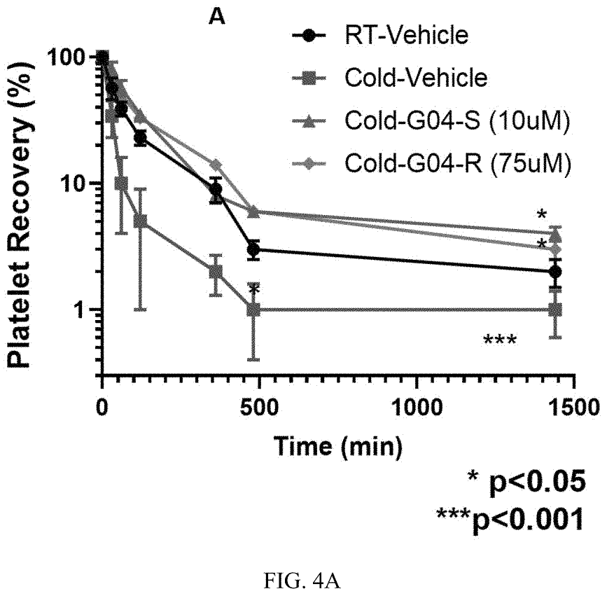

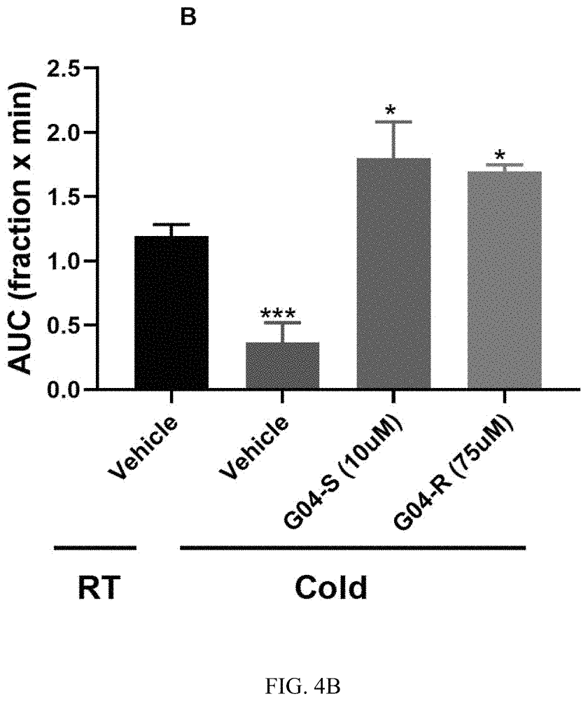

To assess (S)G04 and (R)G04 prevention of platelet activation in vivo, xenotransfusion was performed of long-term (day 7, FIG. 3 and day 14, FIG. 4) stored solutions by taking an aliquot (equivalent to 1×10⁶ platelets) of CFSE-labeled cold-stored human platelets (Acrodose, pooled platelets from four ABO identical donors) that were transfused to conditioned NOD/SCID/gc−/− (NSG) mice and followed for 24 hours post-transfusion.

Four storage conditions were tested. In all storage conditions, the vehicle was the platelet additive solution PAS-3M (Grifols, Barcelona, Spain). The storage conditions varied as follows (1) room temperature storage in vehicle only (RT-Vehicle), (2) cold storage (1-6° C., average ˜2° C.) in vehicle only (Cold-Vehicle), (3) cold storage in vehicle with 10 μM (S)G04 (Cold-G04-S), (4) cold storage in vehicle with 75 μM (R)G04 (Cold-G04-R).

FIG. 3A shows the recovery at different time points of platelets stored for 7 days. FIG. 3B shows the AUC derived from FIG. 3A. *p<0.05; ***p<0.001 between cold-stored-vehicle and RT-vehicle platelets. Data is presented as an average of a triplicate analysis. FIG. 4A shows the recovery at different time points of platelets stored for 14 days. FIG. 4B shows the AUC derived from FIG. 4A. *p<0.05; ***p<0.001 between cold-stored groups and RT vehicle platelets. Data is presented as an average of a triplicate analysis.

Cold-stored platelet survival in vehicle only was significantly reduced as assessed by platelet recovery (FIG. 3A and FIG. 4A) and area under the curve (AUC, FIGS. 3B and 3B). Cold storage of platelets in presence of (S)G04 10 μM or (R)G04 75 μM completely prevented the decreased survival of platelets stored in cold (vehicle) for 7 or 14 days. These data confirm that (S)G04 is a potent inhibitor of cold-stored platelet phagocytosis and in vivo clearance.

In Vivo Clot Formation Studies

Next, the influence of G04 ((S)G04 and (R)G04) on in vivo clot formation was examined. Congenic murine platelets were stored in cold (1-6° C.) for 4 hours and then administered to mice 24 hours after administration of aspirin. Bleeding time was measured for aspirinated mice receiving no platelets, vehicle-only platelets stored at room temperature, vehicle-only cold-stored platelets, vehicle+(R)G04 (75 μM) cold-stored platelets, and vehicle+(S)G04 (10 μM) cold-stored platelets. The results were compared to control non-aspirinated mice (Basal).

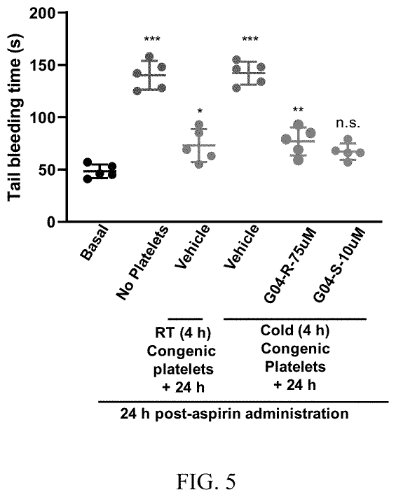

The transfusion of congenic murine platelets in presence of (S)G04 (10 μM) completely corrected the bleeding time of aspirinated mice as of 24 hours post-administration of platelets (FIG. 5; *p<0.05; **p<0.01; ***p<0.001 between baseline and each of the other groups). RT-stored and cold, (R)G04 (75 μM) stored platelets also corrected significantly the bleeding time but not at the extent of (S)G04 (10 μM).

These data demonstrate that Rho GTPase inhibitors (S)G04 or (R)G04 can prevent activation of platelets under a stress condition such as refrigeration, while maintaining their hemostatic activity. This effect is evident in both mouse and human platelets.

1. A composition for platelet storage comprising platelets and a compound represented by the chemical structure of 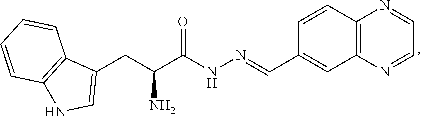 [SMILES: N[C@@H](Cc1c[nH]c2ccccc12)C(=O)N/N=C/c1ccc2nccnc2c1]
or a pharmaceutically acceptable salt thereof;
wherein the compound is present in an amount of about 1 μM to about 50 μM.

2. (canceled)

3. (canceled)

4. (canceled)

5. A composition for platelet storage comprising platelets and a compound represented by the chemical structure of 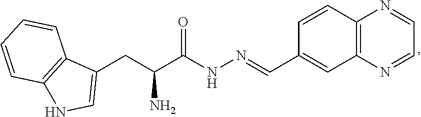 [SMILES: N[C@@H](Cc1c[nH]c2ccccc12)C(=O)N/N=C/c1ccc2nccnc2c1]
or a pharmaceutically acceptable salt thereof;
wherein the (S) form of the compound is present in an enantiomeric excess of at least approximately 94%.

6. A composition for platelet storage comprising platelets and a compound represented by the chemical structure of 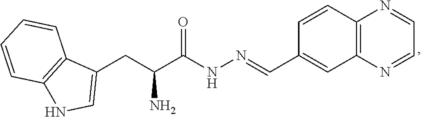 [SMILES: N[C@@H](Cc1c[nH]c2ccccc12)C(=O)N/N=C/c1ccc2nccnc2c1]
or a pharmaceutically acceptable salt thereof;
wherein the amount of said compound in said composition is sufficient to achieve platelet survival and 24-hour recovery greater than about 65% of fresh peripheral blood platelets after 7-21 days of cold storage.

7. The composition of claim 1, wherein the compound is present in an enantiomeric excess of approximately 97%.

8. The composition of claim 1, further comprising a physiologically acceptable carrier.

9. The composition of claim 8, wherein said carrier is a buffer.

10-17. (canceled)

18. A method for storing platelets comprising storing said platelets in a composition according to claim 1.

19. The method of claim 18, wherein platelet survival is greater than about 65% after a storage period of 7-21 days of cold storage.

20. The method of claim 18, wherein the storing is carried out at a temperature from about 0° C. to about 20° C.

21. (canceled)

22. (canceled)

23. (canceled)

24. A method for improving platelet survival upon transfusion comprising:
contacting platelets with a composition of claim 1 or a pharmaceutically acceptable salt thereof; and
infusing said contacted platelets into a subject.

25. The method of claim 24, wherein said compound is present at a concentration from about 1 μM to about 20 μM.

26. The method of claim 24, wherein the platelets are stored in the presence of the compound at a temperature of about 1° C. to about 25° C.

27. The method of claim 24, wherein the platelets are stored in the presence of the compound for a period of about 1 to about 21 days.

28. (canceled)

29. Use of storing platelets in vitro comprising storing said platelets in a composition according to claim 1.

30. The use of claim 29, wherein platelet survival is greater than about 65% after a storage period of 10-14 days of cold storage.

31. The use of claim 29, wherein the storing is carried out at a temperature from about 0° C. to about 20° C.

32. (canceled)

33. (canceled)

34. (canceled)

35. Improving platelet survival for use in a transfusion comprising:
contacting platelets with a composition of claim 1 or a pharmaceutically acceptable salt thereof.

36. The use of claim 35, wherein said compound is present at a concentration from about 1 μM to about 20 μM.

37. The use of claim 35, wherein the platelets are stored in the presence of the compound at a temperature of about 1° C. to about 25° C.

38. The use of claim 35, wherein the platelets are stored in the presence of the compound for a period of about 1 to about 21 days.

39. (canceled)
